(19) United States
(12) Patent Application Publication
(10) Pub. No.: US 2003/0091637 A1
(43) Pub. Date: May 15, 2003

(54) Use of a copolymer to prepare a pharmaceutical form that contains a peptide of protein as active principle

(76) Inventors: Hans-Ulrich Petereit, Darmstadt (DE); Thomas Beckert, Darmstadt (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/239,867

(22) PCT Filed: Oct. 15, 2001
(86) PCT No.: PCT/EP01/11899

(51) Int. Cl. A61K 39/395; A61K 38/43; A61K 38/24; A61K 9/32

(52) U.S. Cl. 424/482; 424/130.1; 424/94.1; 514/2

(57) ABSTRACT

The invention relates to the use, as the coating agent for a pharmaceutical form comprising a core containing a pharmaceutical active principle that is a peptide or a protein, of a copolymer or of a mixture of copolymers of C1 to C4 alkyl esters of acrylic or methacrylic acid, containing methacrylic acid alone or in a proportion of 5 to 25 wt % relative to the mixture. The invention also relates to the corresponding pharmaceutical form itself.

The invention relates to the use of a copolymer for preparation of a pharmaceutical form that contains a peptide or protein as the active principle, as well as to the pharmaceutical form obtained by the said use.

### PRIOR ART

U.S. Pat. Nos. 5,591,433, 5,629,001, 5,783,193 and 6,174,529 B1 describe the oral administration of therapeutically active proteins. Examples of therapeutically active proteins are inoculants (vaccines), proteins for treatment of autoimmune diseases or proteins designed to prevent rejection of foreign tissue in organ transplants. For this purpose the proteins are formulated on cores (nonpareils) together with stabilizing substances such as lactose, mannitol or trehalose, whose purpose is to impart protection during subsequent coating with a polymeric coating agent and during passage through the gastrointestinal tract. Exclusively emulsion polymers formulated under aqueous conditions are used as the polymeric coating agents. Examples of suitable polymers are hydroxypropylmethyl cellulose acetate succinate or EUDRAGIT® L 30 D, a copolymer of 50 wt % of methyl methacrylate and 50 wt % of methacrylic acid. The polymer can be used together with adjuvants such as 0 to 30 wt % of plasticizer, 0 to 3 wt % of talc and 0.0025 wt % of anti-foaming agent, such as silicone or sorbitan sesquioleate. The coating temperatures should range between 30 and 50° C.

The copolymers to be used within the context of the present invention are known from European Patents EP 0704207 A2 and EP 0704208 A2. EP 0704207 A2 describes thermoplastic plastics for pharmaceutical coatings that are soluble in gastric fluids. They are copolymers comprising 16 to 40 wt % of acrylic or methacrylic acid, 30 to 80 wt % of methyl acrylate and 0 to 40 wt % of other alkyl esters of acrylic acid and/or methacrylic acid.

EP 0704208 A2 describes coating agents and binders for pharmaceutical coatings that are soluble in gastric fluids. They are copolymers comprising 10 to 25 wt % of methacrylic acid, 40 to 70 wt % of methyl acrylate and 20 to 40 wt % of methyl methacrylate. The description mentions not only single-layer coatings but also multi-layer coating systems. These can comprise a core containing, for example, a basic or water-sensitive active principle, and can be provided with an insulating layer of another coating material such as cellulose ether, cellulose ester or a cationic polymethacrylate, of the EUDRAGIT® type, for example, including also EUDRAGIT® RS and RL, in which case they are additionally provided with the aforesaid coating that is soluble in gastric fluids.

Example 4 of EP 0704208 A2 describes the release of active principle from pellets containing bisacodyl and coated with a copolymer comprising 70 wt % of methyl acrylate, 20 wt % of methyl methacrylate and 10 wt % of methacrylic acid. At a pH of 6.8, 99% of the active principle contained therein is released in only 45 minutes. Further examples demonstrate the dissolution behavior of glass beads coated with copolymer. Starting from pH 7.0, the curve becomes steeper. In further examples, the release of methylene blue from analogously coated tablets is described. Tablets with a copolymer coating comprising 65 wt % of methyl acrylate, 25 wt % of methyl methacrylate and 10 wt % of methacrylic acid did not dissolve in buffer solution of pH 6.8 after 60 minutes, but disintegrated within 50 minutes at pH 7.5.

Tablets and pellets with a copolymer coating comprising 65 wt % of methyl acrylate, 25 wt % of methyl methacrylate and 10 wt % of methacrylic acid have been described by Petereit et al. (1997) (Conference Abstract, AAPS Meeting in Boston, Nov. 2 to 6, 1997, “Practical experiences with a new anionic methacrylic acid copolymer dispersion containing methyl methacrylate and methyl acrylate as structural monomers”). This type of copolymer releases the active principles only at pH levels of about 7.0 and higher and is therefore suitable for release of active principle in the upper segments of the intestine.

### OBJECT AND ACHIEVEMENT

Coatings of EUDRAGIT® L 30 D, a copolymer comprising 50 wt % of ethyl acrylate and 50 wt % of methacrylic acid, also exhibit actve-principle release which is undesirably premature to at least some extent. This is critical in particular for active principles that are proteins or peptides, since these are then exposed to the action of the proteolytic enzymes present in these segments of the intestine. A further problem for active principles that are proteins or peptides is possible denaturing of their structure. This can occur completely or partly during storage of the pharmaceutical form, due to acid groups present in the polymer coating or to adjuvants such as plasticizers, which are also contained therein.

The object was therefore to provide a pharmaceutical form which is suitable in particular for active principles that are proteins or peptides.

The object is achieved by use, as the coating agent for a pharmaceutical form comprising a core containing a pharmaceutical active principle that is a peptide or a protein, of a copolymer or of a mixture of copolymers of C1 to C4 alkyl esters of acrylic or methacrytic acid, containing methacrylic acid alone or in a proportion of 5 to 25 wt % relative to the mixture.

From the inventive use there is therefore obtained a pharmaceutical form comprising a core containing a pharmaceutical active principle that is a peptide or a protein, and a polymer coating that is a copolymer or a mixture of copolymers of C1 to C4 alkyl esters of acrylic or methacrylic acid, containing methacrylic acid alone or in a proportion of 5 to 25 wt % relative to the mixture.

### OPERATION OF THE INVENTION

The inventive use leads to a pharmaceutical form whose performance in the USP release test, wherein the release of active principle is determined in each case 3.0 hours after test start, is characterized as follows:

at pH 1.2, less than 20%, preferably less than 10% is released,

at pH 6.8, less than 20%, preferably less than 10% is released,

at pH 7.2, 20 to 80%, preferably 35 to 70% is released,

at pH 7.5, 80 to 100%, preferably 90 to 98% is released.

The USP release test (according to USP XXIV, Method B, modified test for “enenteric coated products”) is known to the person skilled in the art. The essential experimental conditions are in particular: paddle method, 100 rpm, 37° C.; pH 1.2 with 0.1 N HCl, pH 6.8, 7.2 or 7.5 in 0.2 M phosphate buffer adjusted with 2 N NaOH or with HCl.

The copolymers to be used according to the invention are known from EP 0704208 A2 and are obtained by radical polymerization, preferably emulsion polymerization of 50 to 68, preferably 60 to 67 wt % of methyl acrylate, 27 to 45, preferably 21 to 32 wt % of C1 to C4 alkyl esters of acrylic or methacrylic acid, and 5 to 20, preferably 8 to 12 wt % of methacrylic acid.

Obviously the content of methyl acrylate is particularly critical. If this is higher than 68 wt %, it favors rapid dissolution of the polymer coatings even at pH levels of around 6.8. which is undesirable. In the range of 50 to 68, preferably 60 to 67 wt % of methyl acrylate, the desired release characteristic is achieved in combination with the equally critical content of 5 to 20, preferably 8 to 12 wt % of methacrylic acid.

The remaining C1 to C4 alkyl esters of acrylic or methacrylic acid that are also present seem to be less critical for the release behavior. Preferred C1 to C4 alkyl esters of acrylic or methacrylic acid are ethyl acrylate, butyl acrylate and butyl methacrylate, and methyl methacrylate is particularly preferred.

For the polymer coating there can also be used a mixture of a neutral copolymer comprising 20 to 40 wt % of ethyl acrylate and 60 to 80 wt % of methyl methacrylate and of a copolymer comprising 25 to 60 wt % of methacrylic acid and 75 to 40 wt % of methyl methacrylate or 75 to 40 wt % of ethyl acrylate, wherein the proportion of methacrylic acid relative to the mixture is 5 to 25 wt %. Such mixtures are known, for example, from EP A 152038 or EP A 208213.

The copolymers to be used preferably have the form of aqueous dispersions with a solid content of, for example, 20 to 50 wt %, and are applied in a manner known in itself by spray-coating of cores or pellets containing active principle.

The weight of the coating can correspond to 5 to 80, preferably 10 to 40 wt % relative to the weight of the core containing the pharmaceutical active principle.

The pharmaceutical form obtained by the said use comprises a core containing a pharmaceutical active principle, which is a peptide or a protein, and a polymer coating, which is a copolymer or a mixture of copolymers of C1 to C4 alkyl esters of acrylic or methacrylic acid, containing methacrylic acid alone or in a proportion of 5 to 25 wt % relative to the mixture.

The pharmaceutical form can be provided with a polymer coating in the form of a copolymer comprising 50 to 68 wt % of methyl acrylate, 27 to 45 wt % of C1 to C4 alkyl esters of acrylic or methacrylic acid as well as 5 to 20 wt % of methacrylic acid.

The pharmaceutical form can further be provided with a polymer coating of a mixture of a neutral copolymer comprising 20 to 40 wt % of ethyl acrylate and 60 to 80 wt % of methyl methacrylate and of a copolymer comprising 25 to 60 wt % of methacrylic acid and 75 to 40 wt % of methyl methacrylate or 75 to 40 wt % of ethyl acrylate.

Adjuvants that are common in pharmaceuticals but not critical for the invention can be incorporated in standard manner.

Cores

Substrates or cores for the coatings are tablets, granules, pellets and crystals of regular or irregular shape. The size of granules, pellets or crystals usually ranges between 0.01 and 2.5 mm, and that of tablets between 2.5 and 30.0 mm. The substrates usually have an active-principle content of 1 to 95% and if necessary also contain further pharmaceutical adjuvants. Standard production methods are direct pressing, pressing of dry, moist or sintered granules, extrusion followed by shaping to rounded form, moist or dry granulation or direct pelleting (for example on plates), or binding of powders (powder layering) on microspheres which do not contain active principle (nonpareils) or on particles containing active principle.

Besides the active principle, the cores can contain further pharmaceutical adjuvants: binders such as lactose, cellulose and derivatives thereof, polyvinylpyrrolidone (PVP), humectants, disintegration promoters, lubricants, disintegration agents, starch and derivatives thereof, sugars, solubilizers or other agents.

The cores can be provided in standard manner with a pharmaceutical active principle, by using an aqueous binder to apply the corresponding active principle in the form, for example, of an active-principle powder, on substrate particles (nonpareils). The active-principle cores (pellets) can be obtained in the desired size fraction (such as 0.7 to 1 mm) by drying and sieving. Among other names, this method is known as “powder layering”.

Pharmaceutical Active Principles

Proteins or peptides constitute a group of organic macromolecules comprising amino acids held together by peptide bonds. The order in which the amino acids are bonded to one another (amino acid sequence) represents what is known as the primary structure of the proteins. When portions of such peptide chains become three-dimensionally interlinked (for example, by formation of hydrogen bonds), they can lead to helix-like structures (α-helix) or to forms resembling pleated sheets (β-pleated-sheet structure), otherwise known as the secondary structure. Other interactions (ionic and hydrophobic interactions as well as compounds containing disulfide bridges) between different regions of a chain produce folding of the polypeptide chain known as tertiary structure. Several chains of the same or different quality can then merge together to form a quaternary structure (as in hemoglobin).

The pharmaceutical active principle can be, for example, an enzyme, a peptide hormone, an immunomodulating protein, an antigen or an antibody.

The pharmaceutical active principle can be a pancreatin, an insulin, a human growth hormone (hGH), a carboplatin, intron A, calcitonin, cromolyn, an interferon, a calcitonin, granulocyte colony stimulating factor (G-CSF), an interleukin, parathyroid hormones, glucagon, pro-somatostatin, a somatostatin, detirelix, cetrorelix, vasopressin, 1-deaminocysteine-8-D-arginine vasopressin, leuprolide acetate or an antigen obtained from grasses or other plants, such as rye, wheat, barley, oats, bermuda grass, horsetail, maple, elm, oak, sycamore, poplar, cedar, horsetail, thistle.

Antibodies are endogenous albumin compounds of the immunoglobins group; they agglutinate together with foreign organic compounds (antigens) that have invaded the body to form a harmless complex. Antibodies are formed in the lymph nodes of the higher animals and of humans. Immunity exists when antibodies are present in sufficient levels or are produced at accelerated rates. If too many antibodies are present, sudden agglutination can lead to apparent allergic symptoms.

The most widely distributed therapeutic use is found for IgG or monoclonal antibodies formed from cells originating from a parent cell.

Dosage Forms

The described (oral) pharmaceutical form can be produced in the form of coated tablets, of a tablet made from pressed pellets or of pellets filled into a capsule of, for example, gelatin, starch or cellulose derivatives.

Standard Pharmaceutical Adjuvants

Standard pharmaceutical adjuvants can be incorporated in the known manner during preparation of the pharmaceutical form. These adjuvants can be contained in the core or in the coating agent.

Dry flowabilitv agents (anti-stickina agents): Dry flowability agents have the following properties: they have large specific surfaces, are chemically inert, are readily free-flowing and are finely divided. By virtue of these properties they lower the tackiness of polymers that contain polar comonomers as functional groups.

Examples of dry flowability agents are:

aluminum oxide, magnesium oxide, kaolin, talc, silica gel (Aerosils), barium sulfate and cellulose.

Release Agents

Examples of release agents are:

Esters of fatty acids or fatty acid amides, aliphatic long-chain carboxylic acids, fatty alcohols and esters thereof, montan or paraffin waxes and metal soaps, while glycerol monostearate, stearyl alcohol, glycerol behenic acid esters, cetyl alcohol, palmitic acid, camauba wax, beeswax, etc. merit special mention. Standard proportions range from 0.05 to 5 wt %, preferably 0.1 to 3 wt % relative to the copolymer.

Further standard pharmaceutical adluvants: Examples in this category are stabilizers, coloring agents, antioxidants, surfactants, pigments, brighteners, etc. They are used mainly as processing aids, and are intended to ensure a reliable and reproducible preparation process as well as long shelf life. Further standard pharmaceutical adjuvants can be present in proportions of 0.001 to 30 wt %, preferably 0.1 to 10 wt % relative to the copolymer.

Plasticizers: The copolymer or the copolymer mixture is preferably formulated without or with at most 10 wt %, for example with 1 to 7 wt % of a plasticizer. Substances suitable as plasticizers usually have a molecular weight of between 100 and 20,000 and contain one or more hydrophilic groups such as hydroxyl, ester or amino groups in the molecule. Suitable substances are citrates, phthalates, sebacates and castor oil. Examples of suitable plasticizers are citric acid alkyl esters, glycerol esters, phthalic acid alkyl esters, sebacic acid alkyl esters, sucrose esters, sorbitan esters, dibutyl sebacate and polyethylene glycols 4000 to 20,000. Preferred plasticizers are tributyl citrate, triethyl citrate, acetyltriethyl citrate, dibutyl sebacate and diethyl sebacate.

### EXAMPLES

EUDRAGIT® FS 30 D=30% dispersion containing a copolymer comprising 65 wt % of methyl acrylate, 25 wt % of methyl methacrylate and 10 wt % of methacrylic acid.

EUDRAGIT® NE 30 D: 30% dispersion containing a copolymer comprising 30 wt % of ethyl acrylate and 60 to 70 wt % of methyl methacrylate.

EUDRAGIT® L 30 D-55: 30% dispersion containing a copolymer comprising 50 wt % of methacrylic acid and 50 wt % of ethyl acrylate.

1. Preparation of Protein-Containing Cores

500 g of placebo pellets were coated in a GPCG 1 fluidized bed apparatus (GLATT Co., Binzen, Germany) with a solution of 9 g of chicken egg albumin (ovalbumin), 45 g of lactose D 80 and 45 g of Collidon 25 in 396 g of water. The spraying rate was 0.7 g/min. The product temperature was maintained between 24 and 26° C. and did not exceed 30° C. during subsequent drying in the apparatus. Thereafter the pellets were mixed with 0.5% silica gel (AEROSIL 200) and dried overnight at room temperature.

2. Coating with an Anionic Polymer that is Soluble at Higher pH

500 g of the ovalbumin pellets from Example 1 were coated in a GPCG 1 fluidized bed apparatus (GLATT Co., Binzen, Germany) with a film-forming spray suspension of 500 g of EUDRAGIT& FS 30 D, 75 g of talc, 8 g of triethyl citrate and 930 g of water. The spraying rate was 4.8 g/min. The product temperature was maintained between 26 and 28° C. and did not exceed 30° C. during subsequent drying in the apparatus. Thereafter the pellets were mixed with 0.5% silica gel (AEROSIL 200) and for 2 hours at 40° C. in the drying oven.

3. Coating with a Mixture of Anionic Polymer and Insoluble Neutral Polymer that Delays Dissolution:

450 g of the ovalbumin pellets from Example 1 were coated in a GPCG 1 fluidized bed apparatus (GLATT Co., Binzen, Germany) with a film-forming spray suspension of 225 g of EUDRAGIT® NE 30 D, 225 g of EUDRAGITO L 30 D-55, 23 g of 0.1 N sodium hydroxide solution, 68 g of talc and 273 g of water. The spraying rate was 1.7 g/min. The product temperature was maintained between 29 and 30° C. and did not exceed 30° C. during subsequent drying in the apparatus. Thereafter the pellets were mixed with 0.5% silica gel (AEROSIL 200) and for 24 hours at 40° C. in the drying oven.

1. The use, as the coating agent for a pharmaceutical form comprising a core containing a pharmaceutical active principle that is a peptide or a protein, of a copolymer or of a mixture of copolymers of C1 to C4 alkyl esters of acrylic or methacrylic acid, containing methacrylic acid alone or in a proportion of 5 to 25 wt % relative to the mixture.

2. The use according to claim 1, characterized in that there is employed a copolymer comprising 50 to 68 wt % of methyl acrylate, 27 to 45 wt % of C1 to C4 alkyl esters of acrylic or methacrylic acid as well as 5 to 20 wt % of methacrylic acid.

3. The use according to claim 1, characterized in that there is employed a mixture of a neutral copolymer comprising 20 to 40 wt % of ethyl acrylate and 60 to 80 wt % of methyl methacrylate and of a copolymer comprising 25 to 60 wt % of methacrylic acid and 75 to 40 wt % of methyl methacrylate or 75 to 40 wt % of ethyl acrylate.

4. The use according to one or more of claims 1 to 3, characterized in that the copolymer or the copolymer mixture is formulated without or with at most 10 wt % of a plasticizer.

5. The use according to one or more of claims 1 to 4, characterized in that the pharmaceutical active principle is an enzyme, a peptide hormone, an immunomodulating protein, an antigen or an antibody.

6. The use according to claim 5, characterized in that the pharmaceutical active principle is a pancreatin, an insulin, a human growth hormone (hGH), a carboplatin, intron A, calcitonin, cromolyn, an interferon, a calcitonin, granulocyte colony stimulating factor (G-CSF), an interleukin, parathyroid hormones, glucagon, pro-somatostatin, a somatostatin, detirelix, cetrorelix, vasopressin, 1-deaminocysteine-8-D-arginine vasopressin, leuprolide acetate or an antigen obtained from grasses or other plants, such as rye, wheat, barley, oats, bermuda grass, horsetail, maple, elm, oak, sycamore, poplar, cedar, horsetail, thistle.

7. The use according to one or more of claims 1 to 6, characterized in that the weight of the coating corresponds to 5 to 80 wt % relative to the weight of the core containing the pharmaceutical active principle.

8. The use according to one or more of claims 1 to 7, characterized in that the copolymer is applied by spraying from a dispersion.

9. A pharmaceutical form comprising a core containing a pharmaceutical active principle that is a peptide or a protein, and a polymer coating that is a copolymer or a mixture of copolymers of C1 to C4 alkyl esters of acrylic or methacrylic acid, containing methacrylic acid alone or in a proportion of 5 to 25 wt % relative to the mixture.

10. A pharmaceutical form according to claim 9, characterized in that the polymer coating has the form of a copolymer comprising 50 to 68 wt % of methyl acrylate, 27 to 45 wt % of C1 to C4 alkyl esters of acrylic or methacrylic acid as well as 5 to 20 wt % of methacrylic acid.

11. A pharmaceutical form according to claim 9, characterized in that the polymer coating has the form of a mixture of a neutral copolymer comprising 20 to 40 wt % of ethyl acrylate and 60 to 80 wt % of methyl methacrylate and of a copolymer comprising 25 to 60 wt % of methacrylic acid and 75 to 40 wt % of methyl methacrylate or 75 to 40 wt % of ethyl acrylate.

12. A pharmaceutical form according to one or more of claims 9 to 11, characterized in that the polymer coating contains no plasticizer or at most 10 wt % of a plasticizer.

13. A pharmaceutical form according to one or more of claims 9 to 12, characterized in that the polymer coating has been applied by spraying from a dispersion.

14. A pharmaceutical form according to one or more of claims 9 to 13, characterized in that the pharmaceutical active principle is an enzyme, a peptide hormone, an immunomodulating protein, an antigen or an antibody.

15. A pharmaceutical form according to claim 14, characterized in that the pharmaceutical active principle is a pancreatin, an insulin, a human growth hormone (hGH), a carboplatin, intron A, calcitonin, cromolyn, an interferon, a calcitonin, granulocyte colony stimulating factor (GCSF), an interleukin, parathyroid hormones, glucagon, prosomatostatin, a somatostatin, detirelix, cetrorelix, vasopressin, 1-deaminocysteine-8-D-arginine vasopressin, leuprolide acetate or an antigen obtained from grasses or other plants, such as rye, wheat, barley, oats, bermuda grass, horsetail, maple, elm, oak, sycamore, poplar, cedar, horsetail, thistle.

16. A pharmaceutical form according to one or more of claims 9 to 15, characterized in that it is produced in the form of tablets, pellets, tablets pressed from pellets or pellets filled into capsules.
